(19) United States
(12) Patent Application Publication
(10) Pub. No.: US 2007/0118397 A1
(43) Pub. Date: May 24, 2007

(54) Monitoring of medical conditions

(76) Inventors: Anthony Williams, Tintenbar (AU); Ronald Grunstein, Bellevue Hill (AU); Gunnar Unger, Kingsgrove (AU)

Correspondence Address: LADAS & PARRY LLP, 224 SOUTH MICHIGAN AVENUE, SUITE 1600, CHICAGO, IL, 60604, US

(21) Appl. No.: 10/556,736

(22) PCT Filed: May 14, 2004
(86) PCT No.: PCT/AU04/00609

§ 371 Date: Feb. 8, 2006

(30) Foreign Application Priority Data
May 14, 2003 (AU) 2003902308

(51) Int. Cl. G06Q 10/00; A61B 5/00

(52) U.S. Cl. 705/2; 600/300

(57) ABSTRACT

The present invention provides a medical device (13) and an associated computing system (12) for performing a medical test a predetermined number of times, before a control means disables the test device, and subsequent analyses of the test.

### FIELD OF THE INVENTION

The present invention relates to a method and system for providing a medical test, and specifically, but not exclusively, to a method and system for providing a sleep related breathing disorder test.

### BACKGROUND OF THE INVENTION

The diagnosis of many medical diseases and disorders generally involves a complex and lengthy study of the affected patient, involving many hours of a physicians time and expertise, in addition to a large amount of expensive, specialised and sophisticated diagnostic equipment.

For example, in diagnosing sleep related breathing disorders, one method of diagnosis involves a formal sleep study in a sleep laboratory after referral from a sleep physician. Such an investigation would usually occur in a hospital or private laboratory environment. Sleep studies are generally complex and expensive because they seek to measure a number of different parameters simultaneously. A “parameter” will be understood to encompass any measurable statistic, such as, for example, a patient's heart rate, blood pressure, breathing pattern, or brain activity.

Moreover, in many countries, such as Australia, sleep physicians must be sufficiently “credentialled” to request such a service. Therefore, such formal sleep studies are expensive, time consuming and not easily accessible to patients.

Furthermore, patients are generally inconvenienced when they are required to undergo such an extensive and intrusive examination. A patient is required to be away from home for a full night, and to sleep in a non-natural environment. The patient may also incur additional cost through the need to visit a specialist for referral and follow-up on several occasions. Travel costs and a potentially negative impact on work and recreation time adds to a detrimental effect on the patient.

Therefore, there has been a trend in recent years to develop simpler, less expensive triage or screening and diagnostic devices that focus on very few parameters and in some cases, a single parameter. These devices have been developed in an attempt to provide opportunities for home based assessment of patients. However, these devices remain quite expensive in terms of capital cost, even though they are less expensive than the equipment required for a complete sleep laboratory.

Patients may still have to travel to a central location to be given instructions on how to use the device, or technicians may have to travel to a patient's home. Therefore, the current devices remain relatively complex and technical failure (particularly with children) can be high when patients do not follow instructions correctly. Due to these drawbacks, doctors are generally reluctant to use and promote such devices.

### SUMMARY OF THE INVENTION

In a first aspect, the present invention provides a device for use in the provision of a medical test, the device comprising,
a testing means arranged to perform the medical test, and
control means arranged to disable the device from performing the medical test once a predetermined number of tests have been performed.

The device may further include storage means arranged to store control data for determining the predetermined number of medical tests that the device is able to perform.

The device may further comprise communication means to enable upload of control data to the device, whereby the device can be enabled to perform a further predetermined number of tests.

The control data may include an encrypted key.

The medical device may further comprise a unique serial number.

The medical device may be an air flow monitoring device for use in a sleep apnoea and/or sleep related breathing disorder test. In this embodiment, the testing means includes,
respiratory sensing means arranged to sense a respiratory signal, the respiratory sensing means being arranged, to communicate with a respiratory tract of a patient, and
memory means arranged to receive and store the respiratory signal.

The respiratory sensing means may be a pressure transducer, and the memory means may be flash memory.

The respiratory sensing means may be in fluid communication with a nasal cannula.

The device may further include a data transfer means, which in this embodiment is compatible with the Universal Serial Bus standard.

In an embodiment including the air flow monitoring device, the nasal cannula may be provided with a coating which is arranged to allow detection of interference with the nasal cannula.

The nasal cannula may further comprise an absorbent pad arranged adjacent to an end portion of the nasal cannula, wherein, in use, the absorbent pad traps cellular matter released by the patient.

In a second aspect, the present invention provides a computing device for use in the provision of a medical test comprising,
means for acquiring medical test information from a medical device,
means arranged to analyse the medical test information; and
means arranged to disable the computing device from performing the analysis once a predetermined number of analyses have been performed.

The computing device may further comprise means for receiving control data from an external source, the control data enabling the computing device to perform a further predetermined number of analyses.

The control data may be received from a central server located on a computer network.

The computing device may further comprises means for uploading control data to the computing device, wherein the uploading of control data enables the computing device to perform a predetermined number of analyses.

The medical device may be a device in accordance with the first aspect of the invention, and the medical test information may be a respiratory signal.

The means for analysing the medical test information may analyse the respiratory signal to determine the presence of an apnoea pattern, and may further correlate the apnoea pattern to a diagnosis.

The means for analysing the respiratory signal may be in the form of a software application.

In a third aspect, the present invention provides a computing system for use in the provision of medical services, the computing system being arranged to communicate remotely with any one of a medical device or a computing system, and comprising,
means for identifying the any one of the medical device or the computing system, and
means to enable the upload of control data to any one of the medical device or the computing system, whereby the any one of the medical device or the computing system can be enabled to perform a predetermined number of medical tests or analyses.

The means for identifying the medical device is a code reading means arranged to read a unique code embedded in the medical device, and the unique code may be a serial number.

The control data may include an encrypted key, wherein the encrypted key contains a numerical value which corresponds to the number of times the any one of the medical device or the computing system may be used before being rendered inoperable.

The encrypted key may contain the unique serial number, wherein the device will only be rendered operable if the unique serial number contained in the encrypted key matches the unique serial number contained in the medical device.

In a fourth aspect, the present invention provides a system for the provision of a medical test, including a medical device in accordance with the first aspect of the present invention, and a computing device in accordance with the second aspect of the present invention.

The system may further include a computing system in accordance with the third aspect of the present invention.

In a fifth aspect, the present invention provides a method of providing a medical service, comprising the steps of
a service provider providing access to a system in accordance with the fourth aspect of the invention to a medical professional,
the service provider enabling operation of any one of the medical device and the computing system for performing a predetermined number of medical tests,
wherein, once the predetermined number of medical tests have been carried out, the any one of the medical device and the computing system is rendered inoperable.

The method may further comprise the step of, in response to a request from the medical professional, the service provider re-enabling the any one of the medical device and the computing system to allow a further predetermined number of medical tests to be carried out.

The request from the medical professional may include the further step of providing payment to the service provider in return for re-enabling the any one of the medical device and the computing system.

In a sixth aspect, the present invention provides a computer program arranged, when loaded on a computing system, to implement a method in accordance with a fifth aspect of the present invention.

In a seventh aspect, the present invention provides a computer readable medium providing a computer program in accordance with a sixth aspect of the invention.

In a eighth aspect, the present invention provides a device for use in the provision of a medical test to a test patient, the device comprising,
a testing means arranged to perform a medical test,
control means arranged to disable the device from performing a medical test once a predetermined number of tests have been performed, and
means for obtaining a DNA sample from the test patient during performance of the medical test.

In a preferred embodiment, the present invention advantageously provides a system by which a medical professional (such as a doctor) may provide a medical service to clients without incurring the associated capital cost inherent in purchasing and maintaining expensive medical equipment.

This advantage is achieved, in part, by providing a system including a medical device which collects patient data and an associated computing system which analyses the data. The system “counts” the number of times a medical test has been performed. When a predetermined value is reached, the system is rendered inoperable, to prevent unauthorised use of the medical device. The system may be rendered inoperable by either disabling the medical device, thereby preventing the collection of further data, or by disabling the computing system, thereby preventing the analysis of further collected data. This allows a manufacturer or supplier of the system to provide (eg. lease) the medical device to a doctor for a nominal value, but requires the doctor to pay a fee for each use of the device.

The system is preferably rendered operable by the introduction of an encrypted key. The encrypted key, in one embodiment, may be downloaded to the medical device from a central server, via a computing device located in a doctor's surgery. In another embodiment, the encrypted key may be downloaded or manually keyed into the computing system. It will be understood that the encrypted key is a preferable feature, and in other embodiments, the medical device may be rendered operable by resetting a digital counter, or by any other suitable means.

The encrypted key is created by the central server, and may use as an input during creation, a unique serial number which is contained in each medical device or in each computing device. This serial number is provided in a suitable readable format. The serial number is used as a “seed” to generate the encrypted key.

In one embodiment, when a doctor wishes to perform a medical test using the medical device, the medical device is connected to the doctor's computing device. The serial number located in the medical device is communicated to the central server in an encrypted format. The central server generates an encrypted key, the encrypted key including information about the medical device (ie. the serial number of the medical device). The encrypted key is then uploaded into the device, which allows the device to perform a predetermined number of medical tests.

In an alternate embodiment, the computing device (rather than the medical device) may be rendered inoperable when analysis of a predetermined number of data sets has been performed.

The encrypted key would be downloaded or keyed into the computing device to render the software application operable, and the medical device would be capable of performing an unlimited number of medical tests.

### DETAILED DESCRIPTION OF THE DRAWINGS

Features and advantages of the present invention will become apparent from the following description of an embodiment thereof, by way of example only, with reference to the accompanying diagrams, in which:

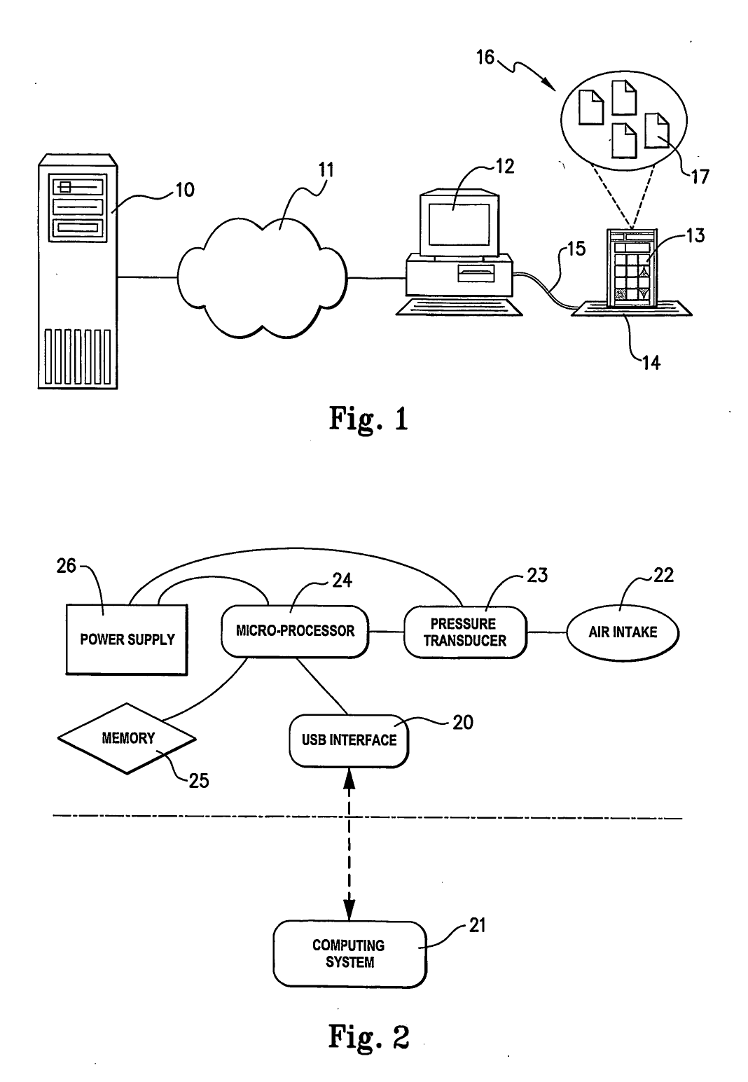

FIG. 1 is a schematic diagram of a system in accordance with an embodiment of the present invention,

FIG. 2 is a schematic block diagram illustrating componentry of a sleep related breathing disorder monitoring device in accordance with an embodiment of the present invention, and

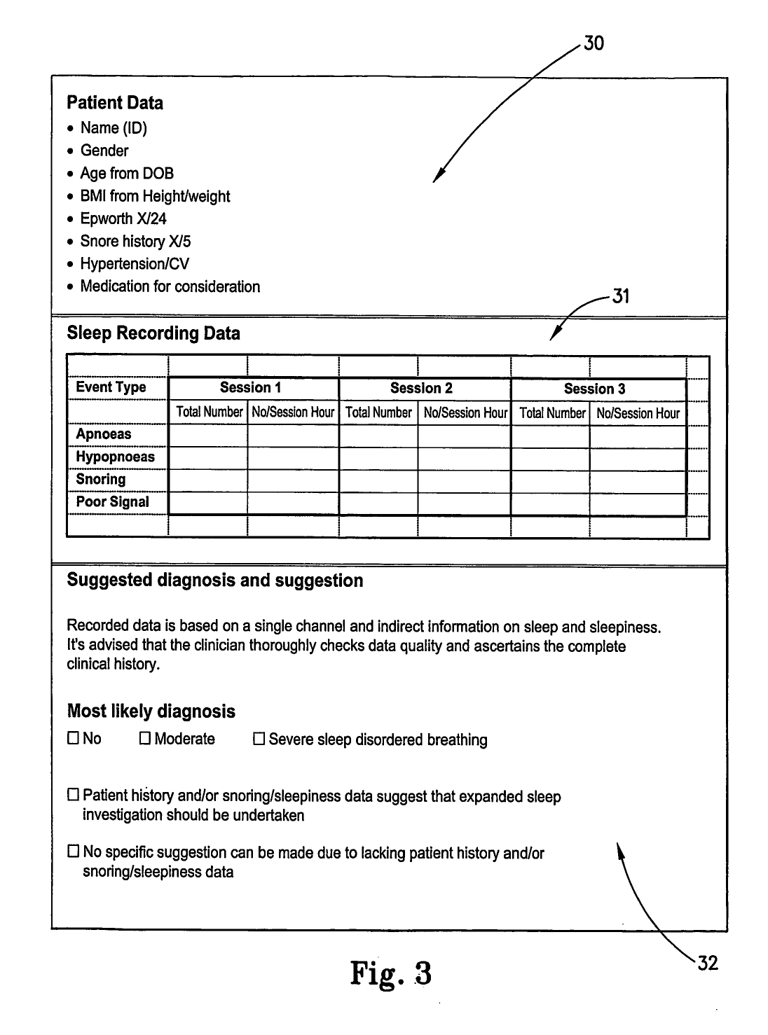

FIG. 3 is a sample report produced by the analysis software in accordance with an embodiment of the present invention.

### DETAILED DESCRIPTION OF A SPECIFIC EMBODIMENT

In a preferred embodiment, the present invention comprises a system including a computing device arranged to interact with a central server, and a medical device arranged to interact with the computing device. Together, the computing device, the central server and the medical device form a system for facilitating provision of medical tests (in one embodiment being a sleep related breathing disorder test) to patients.

In FIG. 1, there is shown a schematic diagram depicting one embodiment of the present invention. A central server 10 is connected to an appropriate network 11, such as the Internet. A doctor accesses the central server via the network 11 from a computing device 12. In the embodiment in accordance with the present invention the computing device 12 is a personal computing device, but the computing device may be any appropriate device, such as a personal digital assistant, or a proprietary computing device arranged to interact solely with the central server 10 and a medical device 13. In the embodiment herein described the medical device 13 is a sleep related breathing disorder monitoring device. The device collects data regarding a patient's breathing pattern (ie. air flow in and out of the nostrils of the patient). However, it will be understood that any suitable parameter data may be attached, such as a pH reading (ie. for monitoring gastric reflux), a heart rate signal (for monitoring possible heart arrhythmia) or an eyelid signal (for vigilance testing). The sleep related breathing disorder device 13 may be attached to the computing device 12 either via a cradle 14 or directly via a USB connection 15. The sleep related breathing disorder monitoring device 13 contains flash memory 16, a portion of which houses an encrypted key 17.

Before the sleep related breathing disorder monitoring device 13 is used by a patient, the encrypted key 17 is modified in flash memory 16, in order to render the device operable. When a predetermined number of medical tests have been carried out by the sleep related breathing disorder device 13, the encrypted key 17 is modified, deleted or otherwise rendered “useless” in a manner such that the sleep related breathing disorder monitoring device 13 is temporarily rendered inoperative.

If the doctor wishes to continue to use the sleep related breathing disorder monitoring device 13, the doctor must retrieve a new encrypted key from the central server 10 to the computing device 12. The encrypted key may subsequently be keyed into the computing device 12 to the sleep related breathing disorder monitoring device 13, at which time the sleep related breathing disorder monitoring device 13 becomes functional.

The computing device in the present embodiment includes a software application. The software application may be provided to and/or installed on the computer by any suitable method, such as via the Internet or on a CD-ROM.

The software application has a number of functions, which may include, but are not limited to, enabling a doctor to set up the sleep related breathing disorder monitoring device for a new patient, testing the sleep related breathing disorder monitoring device to ensure that the sleep related breathing disorder monitoring device is operating correctly, retrieving patient data from the sleep related breathing disorder monitoring device, storing patient data and printing records, connecting directly with the central server (which is generally located at a remote location), monitoring disposable stock requirements and payment plan options, viewing account status, generating tax invoices for payment, and communicating either directly or indirectly with technical and clinical support.

That is, the software application is capable of providing all the standard administrative functions necessary for the successful operation of the sleep related breathing disorder device. It will be understood, however, that the central requirement of the software application will be to analyse data retrieved from the sleep apnoea monitoring device, and any other functions herein described are advantageous but not essential to the software application. Furthermore, such administrative functions, whilst incorporated in the same software application in the embodiment described herein may, in other embodiments, be a separate software application, or a series of stand alone or interrelated software applications.

The core of the software application comprises analysis software routines arranged to analyse respiratory signals provided by the sleep related breathing disorder monitoring device and categorise each breath of a patient according to its shape, height and frequency characteristics. Primarily, the analysis software routines seek to determine whether there is an isolation within a breath.

In the embodiment described, the analysis software routines are incorporated within the software application which resides on the computing device. However, it will be understood that the analysis software routines may, in other embodiments, be provided in a number of different arrangements, such as on a remote server, or embedded in hardware within the sleep related breathing disorder device.

The analysis software routines examine the breath sequence data collected by the sleep related breathing disorder device. The analysis software routines utilise an algorithm that extracts information regarding the frequency and context of certain breath categories. The algorithm performs an analysis of the collected data on the basis of a predetermined series of investigations. For example, if a breathing disorder (such as a cessation of breath during a breath cycle) is detected in a single breath cycle, the algorithm determines the frequency and the distribution of the cessation of breath over the period of the cycle. This process is repeated for each breath cycle captured in the data set.

Once the analysis software routines have processed the entire data set, a series of summary statistics describing each variable of interest is produced. The summary statistics include how many breaths included an apnoea and other parameters related to the existence of sleep related breathing disorders and the distribution of each type of breath over the total time of the recording.

The summary statistics are represented and described in a report that is provided in a format which is understandable to a doctor. That is, the report is written in medical language rather than merely providing numerical data. This translation of raw data to “medical language” is achieved through the use of standard report clauses. The report clauses are chosen from a list of possible report clauses which are pre-programmed into the analysis software. That is, a series of standard scenarios are included in a database. The database uses predetermined rules to select specific report clauses depending on the summary statistics provided by the analysis software routines.

For example, if a first variable (say, a frequency characteristic) falls within a certain range of values, a specific report clause will be selected and printed. As there are limited report clauses available when diagnosing sleep related breathing disorders, all possible combinations of results for each variable are mapped to each of the report clauses available.

The system may also incorporate a facility to allow the summary statistics to be forwarded to a specialist medical professional, or the doctor may provide the report to a specialist medical professional for verification or further analysis.

The system further includes a central server that resides at a location remote from the personal computer and the sleep related breathing disorder monitoring device (although it will be understood that the central server may, in some embodiments, reside in the same area as the computing system).

The central server mirrors the analysis and database software provided on the computing device and may also host a library of clinical reference material. The central server generates the encrypted key to the computing device, which may then be retrieved by the doctor when the doctor logs into the central server.

Each encrypted key may be used as a means of allowing data analysis to occur on the doctor's computing device (in addition to or in place of requiring an analysis key to operate the sleep related breathing disorder device). This provides a variety of methods for allowing the central server to monitor and charge a fee for service provision.

It will be understood that the encrypted key may be obtained in any suitable format. For example, the encrypted key may be directly downloaded from the central server into the computing system or the sleep related breather disorder monitoring device. Alternatively, the key may be provided in an email or on a webpage, and the doctor may subsequently manually enter the key into the software application to allow analysis to proceed.

In one embodiment, the key is an alphanumeric string of data which is “serialised”. That is, the central server generates the key based on the serial number contained in the sleep related breathing disorder monitoring device for the software application. Once this key is entered or downloaded into the software application or the medical device, the software or medical device uses the same algorithm to generate an “expected” key. The expected key is then compared to the received key, and if both keys match the device and/or the software application will be rendered operable.

In some situations, both the computing device and the sleep related breathing disorder device may require complimentary encrypted keys in order to operate, as a safety measure.

In the present embodiment, accounting and stock control functions are initiated by the central server based on individual payment plans and the periodic requirement to purchase an encrypted key, although it will be understood that other methodologies may be developed which allow the delivery of the encrypted key, and such methodologies are within the scope of the present invention.

Furthermore, it will be understood that the encrypted key could be implemented in a number of manners. For example, each encrypted key may only allow one medical test to be performed, such that a plurality of encrypted keys would be downloaded into either the medical device and/or the computing system to allow a plurality of medical tests to be performed. That is, if the doctor wanted to perform five medical tests, he would download five encrypted keys. Alternatively, the encrypted key could include information pertaining to the number of medical tests which may be performed, so that a doctor may purchase one encrypted key which is valid for a number of medical tests.

The central server is also capable of retrieving summary data from the doctor's computing device when the encrypted key is purchased (ie. when a doctor is logged into the central server).

At FIG. 2, there is shown a block diagram illustrating the components of the sleep related breathing disorder monitoring device. As described above, the sleep related breathing disorder monitoring device is comprised of an USB interface 20 which is designed to communicate with a computer 21. The sleep related breathing disorder monitoring device also comprises a suitable air intake device 22 which in this embodiment takes the form of a disposable cannula. Air flow entering the cannula is measured by a pressure transducer 23 to produce an analog signal. The analog signal is converted by a micro-processor 24 to a digital format which is then capable of storage in flash memory 25. The encrypted key is also stored in flash memory 25. Flash memory is used to retain data as it is non-volatile, and capable of information storage without a power requirement. There is also provided a power source 26, which in the present embodiment takes the form of a rechargeable battery. It is also noted that the device may incorporate a visual display unit and necessary controls such that a user may operate the unit (not shown).

The nasal cannula in this preferred embodiment is a disposable nasal cannula to be used in conjunction with the sleep related breathing disorder monitoring device. The nasal cannula is connected directly to the pressure transducer with a secure locking mechanism. Preferably, the length of the nasal cannular tubing is approximately 2 m.

In operation, the embodiment of the present invention allows a doctor to access diagnostic medical equipment at a reduced cost. At a first stage in the process, a doctor accesses a website to purchase or hire a sleep related breathing disorder monitoring device. It will be understood that the sleep related breathing disorder device may be sold as a single unit, in multiple quantities, or as part of a kit (the kit containing other related items such as instruction manuals, spare parts, disposable cannulas, computer software, or any other item which may be related to the satisfactory operation of the device). It will be appreciated that the device or kit may be purchased in any other suitable way. For example, the device or kit may be purchased over the telephone, by faxing an order form to an appropriate retailer, or by purchasing “over the counter”.

In the preferred embodiment of the present invention, the kit includes a customised software application that is downloaded and installed onto the doctor's computing device. The kit may also comprise a user manual, a modified (generally inoperative) demonstration sleep related breathing disorder monitoring device, instructional literature and disposable cannula stock.

It will be appreciated that the components of the kit may be varied as is required for specific circumstances.

The doctor may purchase the kit on a suitable payment plan. This allows the doctor to acquire the kit without incurring any substantial initial cost. In one embodiment, the cost of single kit is covered by approximately ten service episodes, after which the doctor would own the kit outright. However, it is noted that the doctor could choose to pay for the kit up front. That is, the doctor may purchase, lease, or otherwise obtain an embodiment of the present invention by any suitable method, in accordance with local marketing strategies, economic practices and legal considerations.

When a doctor purchases a kit or medical device, the doctor is assigned a unique ID number and account number. Larger practices may also be assigned a practice ID number. In one embodiment, details relating to the number of kits purchased, payment plans, service records, stock purchase and account status will be located in a customised database structure located within the software application that is installed on the doctor's computing device. The details are mirrored on the central server and updated each time the computing device communicates with the central server.

In use, in a consultation, a doctor may decide that the application of a sleep test is necessary. If this is the case, the doctor will provide a patient with a sleep related breathing disorder monitoring device and a simple demonstration in relation to the positioning of the nasal cannular and the operation of the said device. The sleep related breathing disorder monitoring device is then connected via a USB (Universal Serial Bus) connection to the computing device and patient details are entered into the database provided by the software application. A battery and device check is conducted by the computing device. Then, the said device, new cannula, brief questionnaire and instruction packaging is given to the patient to take home.

At home, the patient is able to review the instructions and practice fitting the cannula. Upon retiring for the evening on the designated night, the patient fits the cannula and connects it to the said device as directed. The testing process is initiated by the patient activating a switch on the device. Once the sleep related breathing disorder monitoring device is initiated, the said device collects data regarding the patients breathing pattern (ie. air flow in and out of the nostrils of the patient) as measured by the pressure transducer for a period of eight hours. The data collected is stored in the flash memory. The sleep related breathing disorder monitoring device is arranged to stop automatically. The patient is instructed to wear the said device for the entire night and removes the device upon awakening in the morning. The device may have sufficient memory to allow for recording the breathing pattern of a patient or other parameters for a number of nights.

The patient subsequently returns the sleep related breathing disorder monitoring device to the doctor, and the said device is connected to the doctor's computing device. The data stored in the flash memory is downloaded and analysed by the analysis software. The analysis software analyses the breath cycles data collected by the sleep related breathing disorder monitoring device, and identifies the total number and duration of atypical patterns in the breath cycle, including:

Apnoeas—total cessation of breath within a cycle;

Hypoapneas—smaller amplitude breaths;

Flow limitations;

Oscillation—snoring;

Artefacts—loss of signal, leaks, etc.

The analysis output is coded and stored on the doctor's computing device in a customised database format. Report clause options (from a library of standard report clauses) are retrieved from within the database structure. The report clauses (the selection of which are based on the results of data analysis) and selected summary statistics are presented via a pre-determined report which is assembled by the software, based on the summary statistics. An example report is shown in FIG. 3.

In area 30 there is shown personal patient data including name, date of birth, the patient's body mass index (BMI), plus a short analysis of the patient's historical scores, and a summary of patient data which may be relevant (such as medication history).

In area 31 there is shown a summary of event types and the scores (left blank in the example) for each event type across three separate sleep sessions.

In area 32 there is displayed a series of report clauses which provide a basic analysis (in “medical language”) of the analysed data and provide some guidance to the doctor with regard to both the final diagnosis and the type of corrective action that may be taken to alleviate the disease.

The user interface on the computing device presents the report “on screen” and offers a printed version or a file to copy or save. The report can be explained directly the patient, or can be printed out and sent to the patient, or to the patient in any suitable manner, such as via email.

As the sleep related breathing disorder monitoring device is used with successive patients, the predetermined number of medical tests allowed by the encrypted key is decreased. As the sleep related breathing disorder monitoring device approaches a state of inoperability (due to the performance of the predetermined number of sleep related breathing disorder monitoring tests), the software application residing on the doctor's computing device prompts the doctor to perform a number of tasks.

The doctor is prompted to log into the central server via a website, generate an invoice for the key used to date, receive an additional key (if needed), confirm a standard order of disposable units (ie. nasal cannula), and confirm default delivery arrangements.

The result of these transactions is then added to the account status of the doctor.

Moreover, when the doctor is connected to the central server, the central server is able to harvest summary information in a de-identified format (and other device performance statistics).

This allows the central server operator to perform random quality control testing without the need for specific input from the doctor or the need for the devices to be returned or inspected.

It will be understood that any appropriate industry standard security measures may be adopted to create the encrypted key or any other sensitive data which is transferred to or from the device or to or from the doctor's computing device to the central server. Moreover, in the present embodiment, data is de-identified and no personal patient details are transferred over a public network.

It will be understood that while an embodiment of the present invention communicates via a network such as the Internet, and uses the software application located on the doctor's personal computing device to interact with the central server, any suitable client/server architecture may be employed to implement the invention.

For example, the doctor may log in directly to a central server via a web site interface, and may download an encrypted key directly to the sleep related breathing disorder monitoring device, without the need for the software application. Alternatively, the sleep related breathing disorder monitoring device may have a “built-in” redundancy, wherein the said device is permanently rendered inoperable when the predetermined number of medical tests are performed. In this case, the medical professional is required to purchase a new sleep related breathing disorder monitoring device, rather than downloading a fresh encrypted key.

In an alternate embodiment, the sleep related breathing disorder monitoring device further incorporates an identity checking means arranged to ascertain the identity of a user. This allows the sleep related breathing disorder monitoring device to be utilised in situations where establishing the identity of the user is important.

For example, there may be situations where screening for sleep related breathing disorders is deemed mandatory in order to fulfil licensing conditions (for example, it may become mandatory for truck drivers to take sleep apnoea tests). In such situations, it is important to ensure that the candidate who requires testing (ie. the screening candidate) is in fact the same person who self-administers the test.

An embodiment of the present invention tests the nasal cannula for a DNA profile and matches the DNA profile retrieved from the nasal cannula with requested DNA samples from the screening candidate. The nares section of the cannula is cut from the tubing and stored in a suitable freezer until testing is required. If testing is deemed necessary, a sample of tissue or fluid from the screening candidate (or an existing certified profile) is compared with nasal cell samples retrieved from the nares that have been removed from the cannula. The DNA profile may be obtained from the cells by any suitable method, such as polymerase chain reaction (PCR) analysis. Such a feature creates a deterrent for the screening candidate to manipulate the testing procedure.

Further steps can also be taken to ensure the screening system is not subject to abuse and fraud. To ensure that the screening client does not seek a surrogate candidate, and subsequently clean and reuse the cannula to deposit a legitimate DNA sample on the nares of the cannula, the nares can be stamped with a generic (non-toxic) stable chemical ‘footprint’ to allow identification of any tampering (through the disturbance or absence of the said footprint). Another possibility to prevent tampering is to add a ‘comfort’ pad (made of material that absorbs cell samples—such as felt or gauze) to the nares so that samples can be taken in a manner which precludes easy tampering (since the cellular matter containing DNA is absorbed into the fibres of the pad, which is difficult to clean without displaying evidence of tampering).

The sampling of nasal cells via the cannula also provides further opportunities for health risk assessment. Using nasal squamous cell samples provides other opportunities to detect the progress of sleep disordered breathing, the risk of developing cardiovascular disease, other likelihood of a certain treatment outcomes following say a pharmaceutical treatment or similar.

Whilst an embodiment of the present invention has been described with reference to a sleep related breathing disorder test, it will be understood that an embodiment of the present invention may be employed with any type of medical test and/or any type of medical device, and in particular to any type of medical test that requires the collection of a large sample of data over a defined period of time, such as an ECG (electrocardiogram) or heart signal monitoring test or a temperature monitoring test (ie. monitoring body temperature to determine ovulation time).

Furthermore, it will be understood that the computing system described in the present specification may be provided via the use of any appropriate hardware or software, and elements of an embodiment of the present invention may correspondingly be provided by any appropriate computing hardware or software applications.

In the description above, reference is made to a doctor. However, it will be understood that any suitable medical professional, health care professional or other person may operate or interact with an embodiment of the present invention, including a nurse or a patient.

Modifications and variations as would be apparent to a skilled addressee are deemed to be within the scope of the present invention.

1. A device for use in the provision of a medical test, the device comprising,
a testing means arranged to perform the medical test, and
control means arranged to disable the device from performing the medical test once a predetermined number of tests have been performed.

2. A device in accordance with claim 1, further including storage means arranged to store control data for determining the predetermined number of medical tests that the device is able to perform.

3. A device in accordance with claim 2, the device further comprising communication means to enable upload of control data to the device, whereby the device can be enabled to perform a further predetermined number of tests.

4. A device in accordance with claim 1, wherein the control data is an encrypted key.

5. A device in accordance with claim 1, wherein the medical device further comprises a unique serial number.

6. A device in accordance with claim 1, wherein the medical device is an air flow monitoring device for use in a sleep apnoea and/or breathing disorder test.

7. A device in accordance with claim 1, wherein the testing means includes,
respiratory sensing means arranged to sense a respiratory signal, the respiratory sensing means being arranged, in use, to communicate with a respiratory tract, and
memory means arranged to receive and store the respiratory signal.

8. A device in accordance with claim 7, wherein the respiratory sensing means is a pressure transducer.

9. A device in accordance with claim 7, wherein the memory means is flash memory.

10. A device in accordance with claim 7, wherein the respiratory sensing means is in fluid communication with a nasal cannula.

11. A device in accordance with claim 7, wherein the device further includes a data transfer means.

12. A device in accordance with claim 11, wherein the data transfer means is compatible with the Universal Serial Bus standard.

13. A device in accordance with claim 1, further including a display arranged to display information relating to the operation of the device.

14. (canceled)

15. (canceled)

16. A computing device for use in the provision of a medical test comprising,
means for acquiring medical test information from a medical device,
means arranged to analyse the medial test information, and
means arranged to disable the computing device for performing the analysis once a predetermined number of analyses have been performed.

17. A device in accordance with claim 16, further comprising means for receiving control data from an external source, the control data enabling the computing device to perform a further predetermined number of analyses.

18. A device in accordance with claim 17, wherein the control data may be received from a central server located on a computer network.

19. A device in accordance with claim 18, further comprising means for uploading control data to the medical device, wherein the uploading of control data enables the medical device to perform a predetermined number of medical tests.

20. A device in accordance with claim 16 wherein the medical device is a device in accordance with claim 1.

21. A device in accordance with claim 16, wherein the medical test information is a respiratory signal.

22. A device in accordance with claim 16, wherein the means for analysing the respiratory signal determines the presence of an apnoea pattern.

23. A device in accordance with claim 22, wherein the means further correlates the apnoea pattern to a diagnosis.

24. A device in accordance with claim 22, wherein the means for analysing the respiratory signal may be in the form of a software application.

25. A computing system for use in the provision of medical services, the computing system being arranged to communicate remotely with any one of a medical device or a computing system, comprising,
means for identifying the any one of the medical device or the computing system, and
means to enable the upload of control data to the any one of the medical device or the computing system, whereby the any one of the medical device or the computing system can be enabled to perform a predetermined number of medical tests or analyses.

26. A system in accordance with claim 25, wherein the means for identifying any one of the medical device or the computing system is a code reading means arranged to read a unique code embedded in the any one of the medical device or the computing system.

27. A system in accordance with claim 26, wherein the unique code is a serial number.

28. A system in accordance with claim 25, wherein the control data uploaded to the medical device from the computing system is an encrypted key, wherein the encrypted key contains a numerical value which equals the number of times the medical device may be used before the device is rendered inoperable.

29. A system in accordance with claim 28, wherein the encrypted key contains the unique serial number, and the device will only be rendered operable if the unique serial number contained in the encrypted key matches the unique serial number contained in the medical device.

30. (canceled)

31. A method of providing a medical service, comprising the steps of
a service provider providing access to a system in accordance with claim 25 to a medical professional,
the service provider enabling operation of any one of the medical device and the computing system for performing a predetermined number of medical tests.

32. A method in accordance with claim 31, the method further comprising the step of, in response to a request from the medical professional, the service provider re-enabling the medical device to allow a further predetermined number of medical tests to be carried out.

33. A method in accordance with claim 32, wherein the request from the medical professional includes the step of providing payment to the service provider in return for re-enabling the medical device.

34. A computer program arranged, when loaded on a computing system, to implement a method in accordance with claim 31.

35. A computer readable medium providing a computer program in accordance with claim 34.

36. (canceled)
